(19) United States
(12) Patent Application Publication
(10) Pub. No.: US 2023/0105980 A1
(43) Pub. Date: Apr. 6, 2023

(54) FRAGRANCE DIFFUSER VASE

(71) Applicant: Kenyatta A. Carter, Houston, TX (US)

(72) Inventor: Kenyatta A. Carter, Houston, TX (US)

(21) Appl. No.: 17/495,101

(22) Filed: Oct. 6, 2021

(51) Int. Cl. A61L 9/013; B05B 7/00; B05B 7/24

(52) U.S. Cl. CPC A61L 9/013; B05B 7/0081; B05B 7/2491; A61L 2209/21; A61L 2209/134; A61L 2209/15

(57) ABSTRACT

A fragrance diffuser vase is provided that has variable fan speeds and that separates the plant or flowers from the diffuser while allowing for the unenclosed display of the plant or flowers. Methods of using the fragrance diffuser vase to disperse a scent are also provided.

### FIELD OF THE INVENTION

The present invention relates to fragrance diffusers, particularly to fragrance diffuser vases with variable fan speeds and that separate the flowers from the diffuser while allowing for the unenclosed display of the flowers.

### BACKGROUND

Various fragrance diffuser devices are known that actively or passively diffuse various perfumes, air fresheners, and the like. Passive fragrance diffuser devices are usually based on the spontaneous evaporation of volatile compounds at room temperature. Active fragrance diffuser devices typically include a heater element that heats and evaporates a solid or liquid material containing diffusible volatile compounds.

U.S. Pat. No. 2,807,901 describes a decorative article such as a vase holding artificial flowers. The decorative article comprises a perfumery dispenser designed to be hardly noticeable so as not to mar the attractive appearance of the article and means to control the discharge of the fragrance or fragrance. The decorative article is constructed such that there is no possibility of either the liquid perfume or its volatile emanation coming in contact with the flowers, particularly by entirely enclosing the artificial flowers in a transparent portion of the decorative article.

U.S. Pat. No. 5,547,721 describes a vase that includes a closed base container to receive either a live or imitation plant. A housing in the container has an opening at the base of the vase and houses at least one of: (a) a light; or (b) a scent chemical and blower. When energized, the light is adapted to illuminate through the opening to provide area lighting at the base of the vase. When energized, the blower and the scent chemical drive scented air from the housing to provide proximal air freshening.

Japanese Patent No. 4633797 describes a volatile diffuser that includes two containers, each of which may contain a different material comprising volatile compounds. Each container has at least one opening that is closed with a cap. The device is positioned on a support surface with the containers facing each other in the vertical direction. One container is selected by the user to be in the upper or lower position with respect to the other container, and the other container is configured to be in the opposite position. The opening of the upper container is oriented downward and the opening of the lower container oriented is upward. Each cap allows the volatile compounds contained in the liquid in the containers to pass into the surrounding atmosphere only when the cap is in contact with the liquid. Thus, the upper container is in a volatile emission (“on”) state and the lower container is in a non-volatile (“off”) state. The user selects which active substance to diffuse by rotating the device 180° and placing the “active” container upwards.

U.S. Patent Application Pub. No. 20090071860 describes a two-part vase assembly that includes an outer vase having an open upper end with a peripheral lip, the outer vase being large enough to display decorative items therein. An inner vase is provided that is substantially shorter than the outer vase and is configured to be fit into the upper end of the outer vase. The inner vase is configured and arranged to receive scent diffuser oil and reeds and includes a lip assembly at the upper end thereof. The lip assembly has an outer portion which is configured to removably mate with the peripheral lip of the outer vase, and an inner portion with an inner edge defining an opening which is adapted to receive a stopper element.

A limitation of prior art devices such as those described above is a lack of variable speeds for emitting scents. Another limitation of such prior art devices is that vases like the one described in U.S. Pat. No. 5,547,721 do not allow for a natural and open display of such flowers, and instead completely enclose flowers in a transparent compartment in order to avoid contact between the flowers and the liquid perfume or its volatile emanation.

Accordingly, there is a need for an improved fragrance diffuser system designed to appear like a conventional vase, but with variable fan speeds for emitting scents and that separates the flowers from the diffuser while allowing for the unenclosed display of the flowers.

### SUMMARY

To address the foregoing problems, in whole or in part, and/or other problems that may have been observed by persons skilled in the art, the present disclosure provides compositions and methods as described by way of example as set forth below.

A fragrance diffuser vase is provided, comprising:
a) a container having a substantially closed base, wherein the container defines an interior space and comprises a head portion and a body portion;
b) holes for receiving a plant or flowers disposed in an insert, wherein the insert fits into a ring, wherein the ring fits into the head portion, and wherein the holes are in airflow communication with the interior space and the container's exterior;
c) means for generating a scent disposed in the body portion;
d) a variable speed fan disposed in the body portion to drive the scent from the scent generating means through the holes; and
e) powering means for powering (c) and (d);
   wherein the powering means is configured to be turned on and off by twisting the head portion in relation to the body portion, and wherein the insert separates the plant or flowers from the means for generating a scent while enabling unenclosed display of the plant or flowers. In some aspects, the powering means comprise a power cord and a power cord plug. In other aspects, the head portion is removable.

Methods are also provided for using the fragrance diffuser vases described herein to disperse a scent, comprising loading an essential oil solution into the means for generating a scent and turning on the powering means.

Additional features of the invention will be or will become apparent to one with skill in the art upon examination of the following figures and detailed description. It is intended that all such additional features and advantages be included within this description, be within the scope of the invention, and be protected by the accompanying claims.

### BRIEF DESCRIPTION OF THE DRAWINGS

Having thus described the subject matter of the present invention in general terms, reference will now be made to the accompanying drawings, which are not necessarily drawn to scale, and wherein:

FIG. 1 is a front view of a fragrance diffuser vase in accordance with an embodiment of the present invention;

FIG. 2 is a cross-sectional view of the fragrance diffuser vase shown in FIG. 1;

and

FIG. 3 shows an isometric view of the fragrance diffuser vase shown in FIG. 1.

### DETAILED DESCRIPTION

The subject matter of the present invention now will be described more fully hereinafter with reference to the accompanying drawings, in which some, but not all embodiments of the subject matter of the present invention are shown. Like numbers refer to like elements throughout. The subject matter of the present invention may be embodied in many different forms and should not be construed as limited to the embodiments set forth herein; rather, these embodiments are provided so that this disclosure will satisfy applicable legal requirements. Indeed, many modifications and other embodiments of the subject matter of the present invention set forth herein will come to mind to one skilled in the art to which the subject matter of the present invention pertains having the benefit of the teachings presented in the foregoing descriptions and the associated drawings. All illustrations of the drawings are for the purpose of describing selected versions of the present invention and are not intended to limit the scope of the present invention. Therefore, it is to be understood that the subject matter of the present invention is not to be limited to the specific embodiments disclosed and that modifications and other embodiments are intended to be included within the scope of the appended claims.

### Fragrance Diffuser Vase

The present invention relates to fragrance diffusers, particularly to fragrance diffuser vases with variable fan speeds that separate a plant or flowers from the diffuser while allowing for the unenclosed display of the flowers.

The invention produces a long-lasting scent while also serving as a home decoration. Essential oil diffusion is the process of dispersing essential oils so that their fragrance fills a room or an area with the natural fragrance. A decorative plant or flowers can be placed in the fragrance diffuser vases of the present invention. It is also a goal of the invention to provide relaxation by fragrance therapy without the unsightly appearance of a typical fragrance diffuser. Essential oil fragrances can last six months in one fill without changing the solution.

In one embodiment, a fragrance diffuser vase is provided, comprising:
a) a container having a substantially closed base, wherein the container defines an interior space and comprises a head portion and a body portion;
b) holes for receiving a plant or flowers disposed in an insert, wherein the insert fits into a ring, wherein the ring fits into the head portion, and wherein the holes are in airflow communication with the interior space and the container's exterior;
c) means for generating a scent disposed in the body portion;
d) a variable speed fan disposed in the body portion to drive the scent from the scent generating means through the holes; and
e) powering means for powering (c) and (d);
   wherein the powering means is configured to be turned on and off by twisting the head portion in relation to the body portion, and wherein the insert separates the plant or flowers from the means for generating a scent while enabling unenclosed display of the plant or flowers. In some aspects, the powering means comprise a power cord and a power cord plug. In other aspects, the head portion is removable.

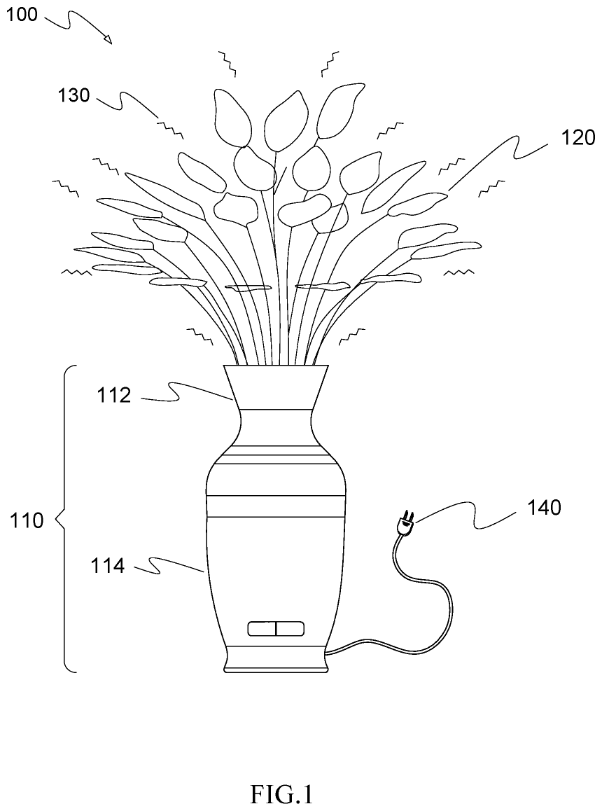

Referring now to FIG. 1, a front view is provided of a plug-in type fragrance diffuser vase 100 in accordance with an embodiment of the invention. Fragrance diffuser vase 100 comprises a container 110 having a substantially closed base, wherein container 100 comprises a head portion 112 and a body portion 114. Holes (not shown) for receiving a plant 120 are disposed in an insert (not shown) that fits into a ring (not shown) that in turn fits into head portion 112, wherein the holes are in airflow communication with the container's interior space and the container's exterior. Means for generating a scent and a variable speed fan (not shown) are disposed in body portion 114 to drive a scent 130 from the scent generating means through the holes.

The fragrance diffuser vase 100 also comprises powering means for powering the variable speed fan and the means for generating a scent, particularly wherein powering means comprises a power cord 142 attached to power cord plug 140. Power cord 142 may be an 18AWG stranded wire or a standard cable size for home use. The powering means is configured to be turned on and off by twisting the head portion 112 in relation to the body portion 114, for example by rotating the head portion 112 about 1 centimeter in one direction to turn the powering means on while rotating the body portion 114 about 1 centimeter in the opposite direction to turn the powering means off.

The means for generating a scent comprise an aroma diffuser. Plug-in type fragrance diffusers like fragrance diffuser vase 100 require 220/230 VAC to start dispersing essential oil. The head portion 112 is removable to place an essential oil solution inside the container 110. In some aspects, the container 110 is about 15 centimeters in width and about 70 centimeters cm in height.

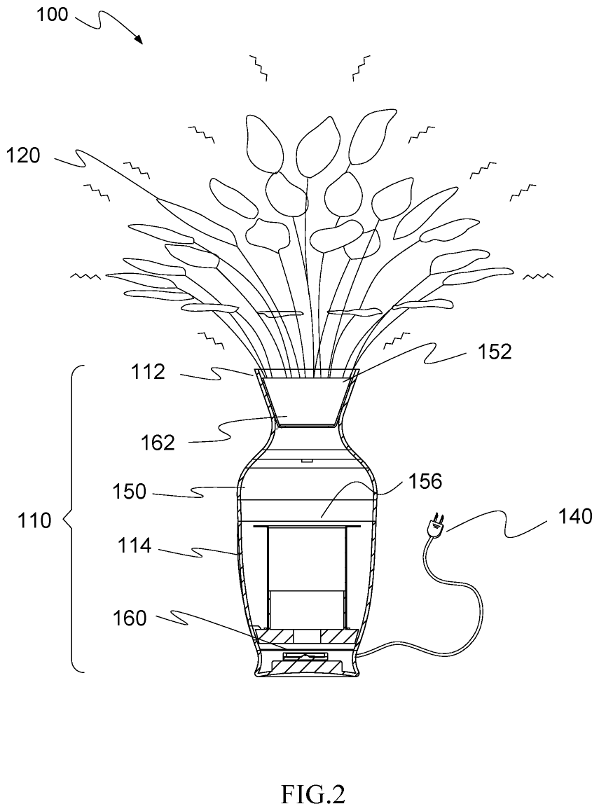

Referring now to FIG. 2, a cross-sectional view is provided of fragrance diffuser vase 100 of FIG. 1, comprising container 110, head portion 112, body portion 114, and plug-in power cord 140. Holes (not shown) for receiving a plant 120 are disposed in insert 162, wherein ring 152 fits into head portion 112 and is configured to receive insert 162, and wherein the holes are in airflow communication with the container's interior space and the container's exterior. Means for generating a scent are disposed in body portion 114 and comprise an essential oil canister 156, a heating element 160, and a variable speed fan 170, which generate scent 130 and drive scent 130 through the holes. Essential oil canister 156 can hold up to 500 milliliters of solution, which can last six months without changing.

The fragrance diffuser vase 100 also comprises powering means for powering variable speed fan 170 and heating element 160, particularly wherein the powering means comprises power cord 142 attached to power cord plug 140. As described above, the powering means is configured to be turned on and off by twisting the head portion 112 in relation to the body portion 114, for example by rotating the head portion 112 about 1 centimeter in one direction to turn the powering means on while rotating the body portion 114 about 1 centimeter in the opposite direction to turn the powering means off.

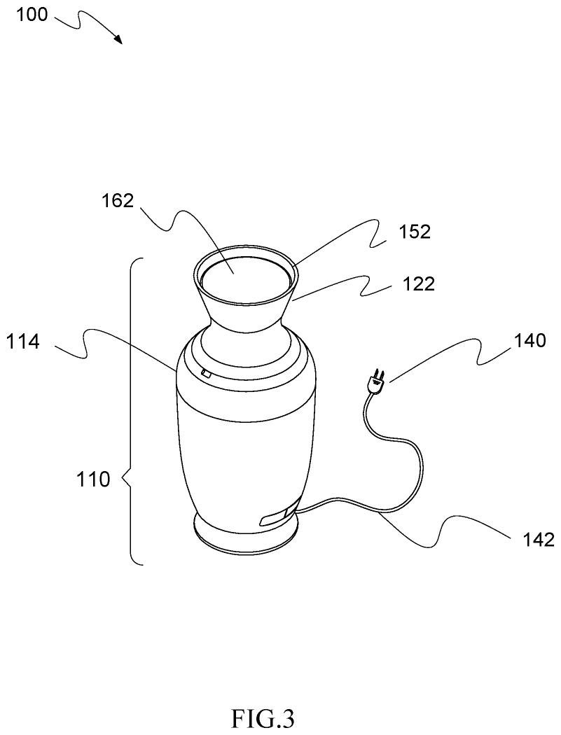

Referring now to FIG. 3, an isometric view is provided of fragrance diffuser vase 100 of FIG. 1, comprising container 110, head portion 112, and body portion 114. Holes 164 for receiving a plant (not shown) are disposed in insert 162, wherein ring 152 fits into head portion 112 and is configured to receive insert 162, and wherein the holes are in airflow communication with the container's interior space and the container's exterior. Powering means comprise a power cord 142 attached to power cord plug 140.

In another embodiment, a method is provided for using any of the fragrance diffuser vases described herein to disperse a scent, comprising loading an essential oil solution into the means for generating a scent disposed in the interior space of the container and turning on the powering means.

### Definitions

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A fragrance diffuser vase comprising:
a) a container having a substantially closed base, wherein the container defines an interior space and comprises a head portion and a body portion;
b) holes for receiving a plant or flowers disposed in an insert, wherein the insert fits into a ring, wherein the ring fits into the head portion, and wherein the holes are in airflow communication with the interior space and the container's exterior;
c) means for generating a scent disposed in the body portion;
d) a variable speed fan disposed in the body portion to drive the scent from the scent generating means through the holes; and
e) powering means for powering (c) and (d);
wherein the powering means is configured to be turned on and off by twisting the head portion in relation to the body portion, and wherein the insert separates the plant or flowers from the means for generating a scent while enabling unenclosed display of the plant or flowers.

2. The fragrance diffuser vase of claim 1, wherein the powering means comprise a power cord and a power cord plug.

3. The fragrance diffuser vase of claim 1, wherein the head portion is removable.

4. A method for using the fragrance diffuser vase of claim 1 to disperse a scent, comprising loading an essential oil solution into the means for generating a scent and turning on the powering means.
